(19) United States
(12) Patent Application Publication
(10) Pub. No.: US 2020/0342964 A1
(43) Pub. Date: Oct. 29, 2020

(54) MEDICAL INFORMATION PROCESSING APPARATUS, ORDERING SYSTEM AND METHOD

(71) Applicant: CANON MEDICAL SYSTEMS CORPORATION, Otawara-shi (JP)

(72) Inventors: Tetsuo SHIMADA, Hachioji (JP); Aina IKEZAKI, Utsunomiya (JP)

(73) Assignee: CANON MEDICAL SYSTEMS CORPORATION, Otawara-shi (JP)

(21) Appl. No.: 16/854,950

(22) Filed: Apr. 22, 2020

(30) Foreign Application Priority Data
Apr. 24, 2019 (JP) 2019-082626

(51) Int. Cl. G16H 10/60; G16H 15/00; G16H 30/40; G16H 50/20

(52) U.S. Cl. CPC G16H 10/60; G16H 50/20; G16H 30/40; G16H 15/00

(57) ABSTRACT

A medical information processing apparatus according to an embodiment includes processing circuitry configured to acquire a possible lesion; before executing a test according to a test order corresponding to the possible lesion, searches previous data for a relevant test item relevant to a test item of the test order; when a result of searching contains the relevant test item, acquire a test result that is collected through a test of the relevant test item, process the acquired test result into a form that matches a purpose of testing of the test order, and output the processed test result; and when the result of searching does not contain the relevant test item, output an additional test order corresponding to the result of searching.

### CROSS-REFERENCE TO RELATED APPLICATIONS

This application is based upon and claims the benefit of priority from Japanese Patent Application No. 2019-082626, filed on Apr. 24, 2019; the entire contents of which are incorporated herein by reference.

### FIELD

Embodiments described herein relate generally to a medical information processing apparats, an ordering system, and a method.

### BACKGROUND

The doctor, the diagnosis and treatment department, and the hospital sometimes change while a patient has a test for multiple times. It is not easy for the current doctor in charge to see what tests were taken on the patient in the past and similar tests are sometimes repeated.

### BRIEF DESCRIPTION OF THE DRAWINGS

FIG. 1 is a block diagram illustrating an exemplary configuration of an ordering system according to a first embodiment;

FIG. 2 is a flowchart for describing a process sequence of the ordering system according to the first embodiment;

FIG. 3 is a flowchart for describing a process sequence of an ordering system according to a second embodiment;

FIG. 4 is a flowchart for describing a process sequence of an ordering system according to a third embodiment;

FIG. 5 is a block diagram illustrating an exemplary configuration of an ordering system according to a fourth embodiment;

FIG. 6 is a flowchart for describing a process sequence to add part information according to the fourth embodiment;

and

FIG. 7 is a flowchart for describing a process sequence of an ordering system according to the fourth embodiment.

### DETAILED DESCRIPTION

With reference to the accompanying drawings, embodiments of a medical information processing apparatus, an ordering system, and a program will be described.

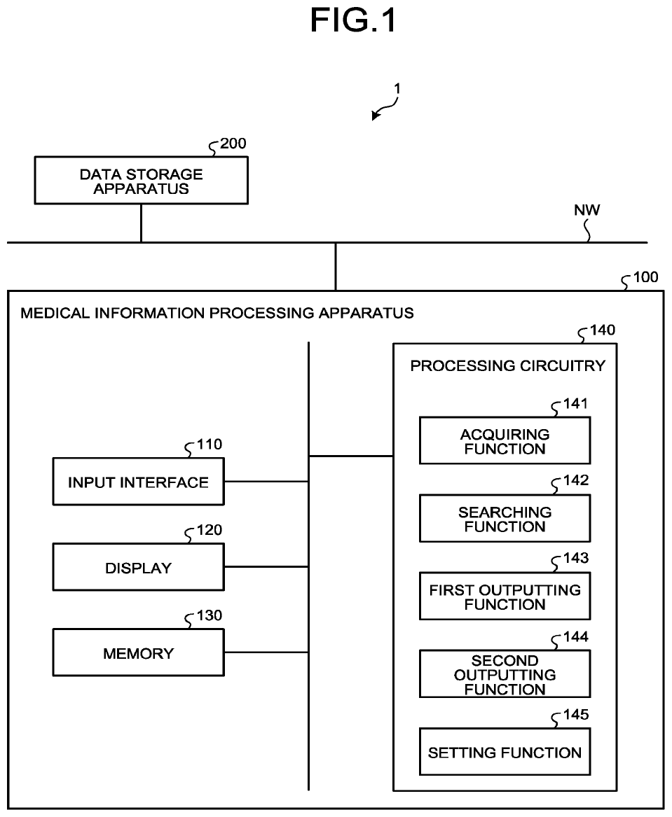

In a first embodiment, an ordering system 1 illustrated in FIG. 1 will be described as an example. FIG. 1 is a block diagram illustrating an exemplary configuration of the ordering system 1 according to the first embodiment.

As illustrated in FIG. 1, the ordering system 1 includes a medical information processing apparatus 100 and a data storage apparatus 200. The medical information processing apparatus 100 and the data storage apparatus 200 are connected such that they can communicate with each other via a network NW. The medical information processing apparatus 100 and the data storage apparatus 200 may be set in the same facility or may be arranged in different facilities, respectively. In other words, the network NW may be formed of a closed local network in a facility or a network via the Internet.

The medical information processing apparatus 100 is an ordering apparatus that outputs a test order corresponding to a possible lesion. Before executing tests according to a test order, the medical information processing apparatus 100 searches previous data that the data storage apparatus 200 stores. In accordance with the result of searching, the medical information processing apparatus 100 outputs at least one of a test result that is collected in the past and an additional test order. The medical information processing apparatus 100 is, for example, implemented using a computer device, such as a work station.

Specifically, as illustrated in FIG. 1, the medical information processing apparatus 100 includes an input interface 110, a display 120, a memory 130 and processing circuitry 140.

The input interface 110 receives various input operations from a user, converts the received input operations into electric signals, and outputs the electric signals to the processing circuitry 140. The user may be a doctor or a co-medical staff, such as a nurse, a pharmacist, a clinical laboratory technologist, radiologist, a physical therapist, a speech therapist, or a dental hygienist. For example, the input interface 110 may be a mouse and a keyboard, a trackball, a switch, a button, a joystick, a touch pad for performing input operations by touching the operation screen, a touch screen with a display screen and a touch pad that are integrated, non-contact input circuitry using an optical sensor, audio input circuitry, or the like.

The input interface 110 may be formed using a tablet terminal, or the like, that can communicated with the medical information processing apparatus 100. The input interface 110 is not limited to one with physical operational parts, such as a mouse and a keyboard. Examples of the input interface 110 include electric signal processing circuitry that receives an electric signal corresponding to an input operation from an external input device that is arranged independently of the medical information processing apparatus 100 and that outputs the electric signal to the processing circuitry 140.

The display 120 displays various types of information. For example, the display 120 displays a test order or a test result. For example, the display 120 displays a graphical user interface (GUI) for receiving various operations from the user. For example, the display 120 is a liquid crystal display or a cathode ray tube (CRT) display. The display 120 may be a desk-top display or may be formed using a tablet terminal that can wirelessly communicate with the medical information processing apparatus 100. The input interface 110 and the display 120 may be integrated with each other. For example, the input interface 110 and the display 120 are implemented using a single touch panel.

The medical information processing apparatus 100 may include a plurality of displays 120. For example, the medical information processing apparatus 100 may include two displays that are physically separate from each other (a dual display) as the display 120. For example, the displays 120 may be controlled such that they relate to each other. For example, the displays 120 are controlled such that they display a continuous single area. In this case, the display area of the display 120 is extended according to the number of displays 120.

The memory 130 is implemented using a random access memory (RAM), a semiconductor memory device, such as a flash memory, a hard disk, or an optical disk. For example, the memory 130 stores programs for respective circuits in the medical information processing apparatus 100 to implement their respective functions.

The processing circuitry 140 executes an acquiring function 141, a searching function 142, a first outputting function 143, a second outputting function 144, and a setting function 145, thereby controlling the entire process performed by the medical information processing apparatus 100.

For example, the processing circuitry 140 reads a program corresponding to the acquiring function 141 from the memory 130 and executes the program, thereby acquiring a possible lesion. For example, the processing circuitry 140 reads a program corresponding to the searching function 142 and executes the program, thereby searching the previous data. For example, the processing circuitry 140 reads a program corresponding to the first outputting function 143 from the memory 130 and executes the program, thereby outputting a test result that was collected in the past according to the result of searching by the searching function 142. For example, the processing circuitry 140 reads a program corresponding to the second outputting function 144 from the memory 130 and executes the program, thereby outputting an additional test order according to the result of searching by the searching function 142. For example, the processing circuitry 140 reads a program corresponding to the setting function 145 from the memory 130 and executes the program, thereby setting a retention period for the test result collected in the past.

The acquiring function 141 is an exemplary acquiring unit. The searching function 142 is an exemplary searching unit. The first outputting function 143 is an exemplary first outputting unit. The second outputting function 144 is an exemplary second outputting unit. The setting function 145 is an exemplary setting unit.

In the medical information processing apparatus 100 illustrated in FIG. 1, each of the processing functions is stored in a mode of a program executable by a computer in the memory 130. The processing circuitry 140 is a processor that reads the programs from the memory 130 and executes the programs, thereby implementing the functions corresponding to the respective programs. In other words, the processing circuitry 140 having read a program has the function corresponding to the read program. FIG. 1 illustrates that the acquiring function 141, the searching function 142, the first outputting function 143, the second outputting function 144, and the setting function 145 are implemented in the single processing circuitry 140. Alternatively, multiple independent processors may be combined to form the processing circuitry 140 and the processors may execute the programs, respectively, thereby implementing the functions. The processing functions of the processing circuitry 140 may be distributed to a single or multiple processing circuits or may be integrated as appropriate and implemented.

The data storage apparatus 200 is a storage that stores previous data. The data storage apparatus 200 is implemented using a computer device, such as a database (DB) server, and stores previous data in a semiconductor memory device, such as a RAM or a flash memory, or storage circuitry, such as a hard disk or an optical disk.

The previous data refers to, for example, a test item that was executed in the past, a test result that was collected through the test of the test item, or information on retention of the test result. For example, the data storage apparatus 200 stores, as the previous data, a test item that was executed in the past and a patient ID indicating the patient on which the test was performed in association with each other.

For example, the data storage apparatus 200 stores the test result as previous data. For example, the data storage apparatus 200 stores, as the previous data, a patient ID representing the patient on which the test was performed and a test result that was collected by the test of the test item in association with each other. In an example, the data storage apparatus 200 stores a test item “X-ray imaging” that was executed in the past, a patient ID representing the patient on which “X-ray imaging” was performed, and image data that was collected through “X-ray imaging” in association with one another.

For example, the data storage apparatus 200 stores the information on retention of the test result as the previous data. For example, the data storage apparatus 200 stores, as previous data, a test item that was executed in the past, a patient ID representing a patient on which the test was performed, and information on retention of the test result that was collected through the test of the test item in association with one another. In an example, the data storage apparatus 200 stores a test item “blood test” that was executed in the past, a patient ID representing a patient on which “blood test” was executed, and information on retention of the blood that was collected through “blood test” (for example, the place where the blood is retained and the period during which the blood is retained) in association with one another. The data storage apparatus 200 may further store a test result. In an example, the data storage apparatus 200 stores a test item “blood test” that was executed in the past, a patient ID representing the patient on which “blood test” was executed, and information on retention of the blood that was collected through “blood test”, and a result of analyzing the blood that was collected through “blood test” (for example, numeric data and views of a doctor, etc.,) in association with one another.

For example, the data storage apparatus 200 is a medical data storage that stores various types of medical data. In an example, the data storage apparatus 200 is an electronic health record storage that is arranged as part of an electronic health record system that is introduced into a hospital, or the like, and that stores various types of vital data that are generated in the electronic health record system. In this case, the data storage apparatus 200 stores various test items, such as measuring a pulse rate, measuring a heart rate, measuring a respiration rate, measuring a blood pressure, measuring a body temperature, and measuring a peripheral oxygen saturation (SpO2), in association with patients on which the tests are performed. The data storage apparatus 200 stores data, such as a pulse rate, a heart rate, a respiration rate, a blood pressure, a body temperature, and a SpO2 that are measured, as test results.

In another example, the data storage apparatus 200 is a specimen test server that stores test data on a specimen, such as blood. The data storage apparatus 200 stores various test items, such as blood drawing and blood analysis, in association with patients on which the tests are performed. The data storage apparatus 200 stores information on a place where a specimen is retained and a period during which the specimen is retained as information on retention of a test result. The data storage apparatus 200 stores processed data, such as a result of analyzing a specimen, as a test result.

In another example, the data storage apparatus 200 is an image storage that stores various types of image data. In an example, the data storage apparatus 200 is a server of a picture archiving communication system (PACS). In this case, the data storage apparatus 200 stores various test items, such as X-ray imaging on the chest, X-ray imaging on a shoulder, and CT on the chest, in association with patients on which the tests are performed. The data storage apparatus 200 stores, as test results, data collected by capturing images of a patient, image data that is generated by executing image generation processing on the data that is collected by capturing images of the patient, data of the result of analyzing the image data, etc.

For example, the data storage apparatus 200 stores the data collected by capturing images of a patient using an X-ray diagnostic apparatus. Data that is collected by capturing images of a patient using a medical image diagnostic apparatus, such as an X-ray diagnostic apparatus, and that is before image generation processing is performed is collectively referred to as raw data. Specifically, first of all, the X-ray diagnostic apparatus applies X-rays to the patient from an X-ray tube and detects the X-rays having passed through the patient with an X-ray detector. The X-ray diagnostic apparatus generates raw data based on a detection signal that is received from the X-ray detector and causes the data storage apparatus 200 to store the generated raw data as a test result. The X-ray diagnostic apparatus executes various types of image generation processing on the raw data, thereby generating X-ray image data. The X-ray diagnostic apparatus causes the data storage apparatus 200 to save the generated X-ray image data as a test result. The X-ray diagnostic apparatus causes the data storage apparatus 200 to store the result of analyzing the generated X-ray image data as a test result. Note that, for example, data on measuring a diameter of a blood vessel and a result of calculating a blood flow are taken as the result of analyzing X-ray image data.

The setting function 145 may set a retention period for various test results. For example, the results of tests, such as X-ray imaging and CT, (raw data and image data) are accumulated daylily and reduces the area where data can be retained in the data storage apparatus 200. The setting function 145 thus sets retention periods for these sets of data, thereby enabling a reduction in the load on the data storage apparatus 200. Specimens, such as blood, are also accumulated daylily and reduces the space of a facility where the specimens are retained. For this reason, the setting function 145 sets a period during which the specimens are retained, thereby enabling a reduction in the load on the facility where specimens are retained.

FIG. 1 illustrates only the single data storage apparatus 200. Alternatively, the ordering system 1 may contain a plurality of data storage apparatuses 200. For example, the ordering system 1 may include the data storage apparatus 200 serving as an electronic health record storage, the data storage apparatus 200 serving as a specimen test server, and the data storage apparatus 200 serving as a PACS server.

The data storage apparatuses 200 may be arranged over a plurality of diagnosis and treatment departments. For example, each of the diagnosis and treatment departments includes at least one data storage apparatus 200 and individually manages previous data. The data storage apparatuses 200 may be arranged over multiple facilities. For example, each of the facilities holds at least one data storage apparatus 200 and individually manages previous data. A facility is, for example, a medical facility, such as a hospital, a clinic, a care facility, or a medical check-up facility. Part of or all previous data may be stored in the memory 130.

The entire configuration of the ordering system 1 according to the first embodiment has been described. With the configuration, the ordering system 1 according to the first embodiment effectively utilizes previous test results. Processes performed by the ordering system 1 according to the first embodiment will be described below.

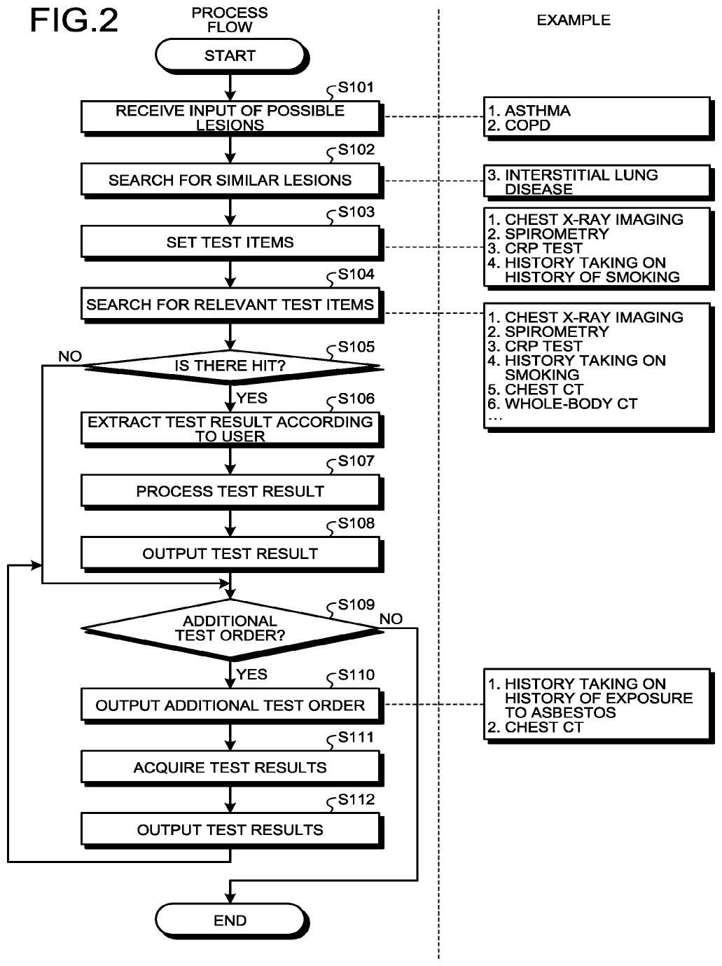

First of all, using FIG. 2, an exemplary procedure taken by the ordering system 1 according to the first embodiment will be described with specific examples. FIG. 2 is a flowchart for describing a process sequence of the ordering system 1 according to the first embodiment.

For example, the acquiring function 141 receives an input of a possible lesion or multiple lesions from a user via the input interface 110 (step S101). For example, the user inputs asthma and chronic obstructive pulmonary disease (COPD) as possible lesions based on the symptom of the patient “coughing”.

When multiple possible lesions are input, the user may take the levels of significance and a prevalence of each of the possible lesions into consideration and input the possible lesions with priorities added thereto. For example, the user adds a priority 1 to asthma and adds a priority 2 to COPD. In other words, as illustrated in FIG. 2, the user inputs “1. asthma” and “2. COPD” as possible lesions.

The acquiring function 141 then searches for a similar lesion similar to the possible lesion that is input by the user (step S102). The similar lesion is, for example, a lesion representing symptoms similar to those of the input possible lesion or a lesion of which cause is similar to that of the input possible lesion. The acquiring function 141 adds the similar lesion contained in a searching result as a possible lesion.

When multiple possible lesions are input, the acquiring function 141 may search for similar lesions based on common characteristic between the input possible lesions. For example, by referring to a database to which characteristics of each lesion are input in advance, the acquiring function 141 specifies common characteristics between the possible lesions and searches for a similar lesion based on the specified characteristics. The database to which the characteristics of each lesion are input in advance is, for example, a diagnostic protocol that is backed by the evidences disclosed with respect to each lesion by related associations. In another example, the database to which characteristics of each lesion are input in advance is a database that is updated as appropriate by a doctor who refers to papers on each lesion. In general, the above-described diagnostic protocol has high reliability and thus is preferably used. On the other hand, a database that is updated as appropriate by a doctor is updated at a rate higher than that of the above-described diagnostic protocol and thus is advantageous in reflecting the latest information.

Alternatively, the acquiring function 141 may search for a similar lesion using a trained model that has learned relevancy between multiple lesions. The trained model is generated before the acquiring function 141 searches for a similar lesion and it stored in, for example, the memory 130. For example, the acquiring function 141 generates a trained model in advance by executing machine learning using multiple lesions as input-side data and lesions that are set by a doctor, or the like, as ones similar to the lesions and stores the trained model in the memory 130. When an input of multiple possible lesions is received from the user, the acquiring function 141 inputs the possible lesions to the trained model that is read from the memory 130. The trained model outputs a lesion similar to the input possible lesions.

For the machine learning performed by the acquiring function 141, various methods, such as deep learning, logistic regression analysis, non-linear discriminant analysis, a support vector machine (SVM), a random forest, and Naive Bayes, can be employed. The acquiring function 141 is described as one that generates the trained model. Alternatively, the trained model may be generated by a apparatus different from the medical information processing apparatus 100. For example, the acquiring function 141 acquires the trained model that is generated by another apparatus via the network NW and stores the acquired trained model in the memory 130.

For example, the acquiring function 141 acquires interstitial lung disease as a similar lesion similar to asthma and COPD and adds the interstitial lung disease as a possible lesion. The acquiring function 141 may add a priority to the possible lesion to be added. For example, the acquiring function 141 adds, to interstitial lung disease, a priority 3 that is lower than those of asthma and COPD and that is input by the user. In other words, as illustrated in the example in FIG. 2, the acquiring function 141 adds “3. interstitial lung disease” as a possible lesion.

The searching function 142 sets test items based on the possible lesions that are acquired by the acquiring function 141 (step S103). In an example, the searching function 142 searches a test list based on the possible lesions, thereby setting a test item. The test list is a list in which a test item(s) necessary for diagnosis is set for each lesion. The test list is, for example, created by a doctor in advance and stored in the memory 130. When there are multiple possible lesions, redundant test items may be omitted as appropriate. For example, the searching function 142 searches the test list with respect to each of the possible lesions “1. asthma”, “2. COPD” and “3. interstitial lung disease”, thereby setting chest X-ray imaging, spirometry (lung function test), C-reactive protein (CRP) test, and history taking on a history of smoking. It has been described that test items are set based on the test list. Alternatively, the searching function 142 may set test items based on an input operation from the user.

When multiple test items are set, the searching function 142 may add priorities to the respective test items. For example, the searching function 142 adds a priority 1 to the chest X-ray imaging, adds a priority 2 to spirometry, adds a priority 3 to CRP test, and adds a priority 4 to history taking on a history of smoking. In other words, as illustrated in the example in FIG. 2, “1. chest X-ray imaging”, “2. spirometry”, “3. CRP test” and “4. history taking on history of smoking” are set as test items.

In setting a priority in each test item, various factors, such as characteristics of each test item (invasiveness, utility of diagnostic result determination, costs, etc.) and the level of urgency of the patient, can be taken into consideration. For example, for a critical care patient, a high priority is added to a test item with high utility such that a diagnosis is enabled by a single or a small number of tests. When the patient is not a critical care patient, a high priority is added to a test item with low invasiveness in order to reduce the stress on the patient.

The test items that are set are output as a test order generally. A test practitioner, such as a doctor or a technologist, carries out tests according to the test order and the doctor makes a diagnosis based on the results of the tests. As for the tests of the test items that are set herein, however, similar tests might have been carried out. For example, while “1. chest X-ray imaging” is set as a test in FIG. 2, chest X-ray imaging might have been already performed on the same patient by another doctor, in another diagnosis and treatment department, or in another hospital. Alternatively, even if chest X-ray imaging has not been performed, X-ray imaging on a part neat the chest or chest imaging with another type of modality might have been already performed.

Under the circumstances, the searching function 142 searches the previous data for a relevant test item relevant to the test items of the test order before executing the tests according to the test order (step S104). The test order may be output before the searching function 142 searches for a relevant test item or is not necessarily output. In other words, as long as the test items of the test order are set, the searching function 142 is able to search for a relevant test item relevant to the test items of the test order regardless whether the test order is issued.

First of all, a relevant test item will be described. A relevant test item is a test item that has relevance with the test items of the test order that are set according to the possible lesions. For example, in FIG. 2, “1. chest X-ray imaging”, “2. spirometry”, “3. CRP test” and “4. history taking on history of smoking” are set as test items of a test order. In this case, the relevant test items include a test item relevant to the test items in addition to “1. chest X-ray imaging”, “2. spirometry”, “3. CRP test” and “4. history taking on history of smoking”.

For example, as illustrated in the example in FIG. 2, the relevant test items include “5. chest CT” and “6. whole-body CT” relevant to “1. chest X-ray imaging”. In other words, because, even when there is no chest X-ray image, chest CT image data or CT image data containing chest CT image data may enable a diagnosis, the searching function 142 incorporates “5. chest CT” and “6. whole-body CT” into relevant test items and perform searching.

A relevant test item can be set, for example, according to a test item list that determines relevance between test items. The test item list is, for example, created in advance by a doctor and is stored in the memory 130. For example, by searching the test item list for each of “1. chest X-ray imaging”, “2. spirometry”, “3. CRP test” and “4. history taking on history of smoking”, the searching function 142 extracts a test item relevant to at least one of the test items as a relevant test item.

For example, as illustrated in FIG. 2, the searching function 142 searches the previous data for relevant test items, such as “1. chest X-ray imaging”, “2. spirometry”, “3. CRP test”, “4. history taking on history of smoking”, “5. chest CT” and “6. whole-body CT”. In other words, the searching function 142 checks whether there is a history relevant to the test items to be performed for this time. The searching function 142 determines whether there is a search hit (step S105). In other words, the searching function 142 determines whether the result of searching contains a relevant test item.

When the result of searching by the searching function 142 contains a relevant test item (YES at step S105), the first outputting function 143 acquires a test result that was collected in the past through the test of the relevant test item and extracts part of the test result according to the user (step S106).

Specifically, first of all, the first outputting function 143 acquires a test result that was collected in the past through the test of a relevant test item contained in the searching result. For example, when the searching result contains the relevant test item “1. chest X-ray imaging”, the first outputting function 143 acquires X-ray image data that was collected through chest X-ray imaging from the data storage apparatus 200 that is the PACS server. In an example, the first outputting function 143 acquires, from the data storage apparatus 200, chest X-ray image data to which the same patient ID is added as additional information together with various types of information, such as the test date, etc. The first outputting function 143 then extracts part of the acquired test result according to the user. Specifically, the first outputting function 143 extracts part of the test result such that parts unnecessary for the current diagnosis are omitted to allow the user to easily refer to the test result. In an example, when the acquired chest X-ray image data is obtained in the department of cardiovascular internal medicine, a result of analysis by computer-aided diagnosis (CAD) on the heart and a result of measuring blood vessel diameters, etc., may be added to the X-ray image data. When the user is, for example, a doctor of the department of respiratory medicine, detailed information on the heart tends to be unnecessary. The first outputting function 143 thus extracts only part pf the acquired X-ray image data excluding the detailed information on the heart. For example, the first outputting function 143 omits the CAD analysis result and the blood vessel diameter measurement result among the acquired X-ray image data and extracts only the basic information, such as the patient ID and the imaging date, and the image information. In this manner, the first outputting function 143 reduces the amount of information and allows the user to easily refer to the test result.

The first outputting function 143 processes the test result that is extracted according to the user into a form that matches the purpose of testing of the current test order. Specifically, the first outputting function 143 processes the test result such that the user easily refers to the test result. For example, when the acquired chest X-ray image data is obtained in the department of cardiovascular internal medicine, the X-ray image data tends to be processed such that the data is suitable for observation of organs of circulation. In an example, processing enabling easy check of the heart and blood vessels by sight is sometimes performed on the X-ray image data that is obtained in the department of cardiovascular internal medicine.

Under the circumstances, the first outputting function 143 processes the X-ray image data into a form that matches the current purpose of testing. For example, when the current purpose of testing is making a diagnosis on the lungs, the first outputting function 143 processes the X-ray image data such that the data is suitable for observation of the lungs. In an example, the first outputting function 143 adjusts the window level (WL) and the window width (WW) of the X-ray image data such that the lungs are easily checked by sight.

It is described herein that X-ray image is acquired. Alternatively, the first outputting function 143 may acquire raw data that is before the image generation processing is performed. For example, the first outputting function 143 may acquire, from the data storage apparatus 200, raw data that is collected by capturing images of a patient with the X-ray diagnostic apparatus instead of X-ray image data. In this case, the first outputting function 143 execute the image generation processing and various types of image processing on the raw data to generate X-ray image data that is processed such that the lungs can be checked easily by sight.

The data storage apparatus 200 may store both X-ray image data and raw data. In such a case, the first outputting function 143 acquires both the X-ray image data and raw data and adds priorities to the X-ray image data and raw data, respectively, and processes them into a form that matches the purpose of testing. In other words, the first outputting function 143 acquires multiple test results that were collected in the past, adds priorities to the acquired test results, respectively, and processes the test results into a form that matches the purpose of testing.

In an example, the first outputting function 143 adds a priority “1” to the raw data, adds a priority “2” to the X-ray image data, and processes each of the raw data and the X-ray image data into a form that matches the purpose of testing. In other words, as for the X-ray image data that is acquired from the data storage apparatus 200, image processing corresponding to a different purpose of testing might have been performed and part of information contained in the raw data might have lacked. For example, when the WL and WW of the X-ray image data are adjusted, information outside the window area may lack. On the other hand, such a lack does not occur in the raw data. Accordingly, when data is processed into a form matching the current purpose of testing, processing based on the raw data often enables generation of more suitable data compared to the case where the data is processed based on the X-ray image data. For this reason, adding a higher priority to the raw data than that to the X-ray image data is preferable.

“1. chest X-ray imaging” is described herein as an example, and other relevant test items can be described similarly. For example, when the result of searching by the searching function 142 contains the relevant test items “2. spirometry”, “3. CRP test” and “4. history taking on history of smoking”, the first outputting function 143 acquires the test results that were collected in the past by the tests of the relevant test items and extracts part of the acquired test results according to the user. The extraction processing may be omitted as appropriate. The first outputting function 143 processes the test result into a form that matches the purpose of testing of the current test order.

The first outputting function 143 outputs the processed test results (step S108). For example, when the user is a doctor of the department of respiratory medicine, the first outputting function 143 creates a display screen on which the test results are displayed based on a format that is generally used in the department of respiratory medicine and displays the display screen on the display 120. When the result of searching by the searching function 142 does not contain any relevant test item (NO at step S105), the above-described steps S106 to S108 are omitted.

When the acquiring function 141 acquires multiple possible lesions, the first outputting function 143 may output a test result for each of the possible lesions at step S108. For example, the first outputting function 143 divides the test result used for diagnosis of “1. asthma”, the test result used for diagnosis of “2. COPD”, and the test result used for diagnosis of “3. interstitial lung disease” into groups and display the grouped test results.

At step S108, the first outputting function 143 may further display the prevalence of each of the possible lesions that are acquired by the acquiring function 141. The prevalence may be general statistical information or may be a possibility that is calculated for each patient. For example, the first outputting function 143 acquires a percentage of the number of cases to a population as the prevalence from the database and displays the prevalence on the display 120. For example, the first outputting function 143 calculates a prevalence for each patient and each lesion based on the test results that are acquired from the previous data and displays the prevalences on the display. In an example, the first outputting function 143 calculates, for the current patient, a possibility of asthma, a possibility of COPD, and a possibility of interstitial lung disease and displays the possibilities on the display 120.

The second outputting function 144 then determines whether to create an additional test order (step S109). The second outputting function 144 may determine necessity of an additional test order automatically or according to an input operation from the user.

For example, at step S105, when it is determined that the result of searching by the searching function 142 does not contain any relevant test item, the second outputting function 144 automatically determines that an additional test order is necessary. For example, at step S105, when it is determined that the result of searching by the searching function 142 contains a relevant test item but it is insufficient, the second outputting function 144 automatically determines that an additional test order is necessary.

In an example, when the searching result contains “2. spirometry”, “3. CRP test”, “4. history taking on history of smoking” and “5. chest CT” out of the relevant test items represented in FIG. 2, the second outputting function 144 determines that no additional test order is necessary. In other words, even when the searching result does not contain “1. chest X-ray imaging” and “6. whole-body CT”, it is often possible to make a diagnosis when there is a test result of “5. chest CT” and thus the second outputting function 144 determines that no additional test order is necessary.

In another example, when the searching result contains “1. chest X-ray imaging”, “3. CRP test” and “4. history taking on history of smoking” among the relevant test items represented in FIG. 2, the second outputting function 144 determines that an additional test order is necessary. In other words, when no test of “2. spirometry” was performed in the past, it is not easy to replace the test with another test result and thus the second outputting function 144 determines that “2. spirometry” is necessary as a test item of an additional test order.

When a determination on whether an additional test order is necessary at step S109 is executed prior to outputting the test result at step S108 and it is determined that an additional test order is necessary, outputting the test result at step S108 may be omitted. In this case, the test result that is processed at step S107 is output at step S112 together with a test result resulting from an additional test order to be described below.

Alternatively, the second outputting function 144 may receive an operation of choosing whether to make an additional test order via the input interface 110 from the operator who has referred to the test result that is output at step S108. In an example, when the searching result contains “1. chest X-ray imaging”, “3. CRP test” and “4. history taking on history of smoking” among the relevant test items represented in FIG. 2, the second outputting function 144 notifies the user of the fact that no test of “2. spirometry” was performed in the past and determines that an additional test order is necessary based on an input operation from the user.

When it is determined that an additional test order is necessary (YES at step S109), the second outputting function 144 creates and outputs an additional test order (step S110). For example, when the result of searching by the searching function 142 does not contains any relevant test item, the second outputting function 144 creates and outputs an additional test order for executing the test items (chest X-ray imaging, spirometry, CRP test, and history taking on the history of smoking) that are set at step S103.

When the result of searching by the searching function 142 contains a relevant test item but it is insufficient, the second outputting function 144 creates and outputs an additional test order for executing a test item, among the test items that are set at step S103, whose corresponding relevant test item relevant to the test item is not contained in the searching result. In an example, when the result of searching by the searching function 142 contains “3. CRP test”, “4. history taking on history of smoking” and “5. chest CT”, while “1. chest X-ray imaging” and “2. spirometry” among the test items that are set at step S103 lack, “1. chest X-ray imaging” is replaceable with the test result of “5. chest CT”. In other words, as for “1. chest X-ray imaging”, the searching result contains a relevant test item relevant to “1. chest X-ray imaging”. On the other hand, as for “2. spirometry”, the searching result does not contain any relevant test item relevant to “2. spirometry”. Accordingly, the second outputting function 144 creates and outputs an additional test order for executing “2. spirometry”.

In another example, when the searching result contains “1. chest X-ray imaging”, “2. spirometry”, “3. CRP test” and “4. history taking on history of smoking” among the relevant test items represented in FIG. 2, the first outputting function 143 processes the test results of the relevant test items into a form that matches the current purpose of testing and represents the processed test results to the operator. When a shade of plaque is confirmed in the chest X-ray image data but there is not smoking history, a possibility of COPD due to exposure to asbestos is suspected. Thus, in order to examine the possibility of COPD due to exposure to asbestos, history taking on the history of exposure to asbestos and chest CT are input as test items of an additional test order. Alternatively, the second outputting function 144 may propose the items of the additional test order to the user.

The second outputting function 144 may add priorities to the respective multiple test items of the additional test order and output the additional test order. For example, as illustrated in FIG. 2, the second outputting function 144 may add priorities to the respective test items and output “1. asthma” and “2. chest CT” as the additional test order. For setting a priority in the each test item, various factors, such as invasiveness, utility and costs of each test item and the level urgency of the patient, can be taken into consideration. For example, for a critical-care patient, a high priority is added to a test item with high utility such that a diagnosis is enabled by a single or a small number of tests. When the patient does not a critical care patient, a high priority is added to a test item with low invasiveness in order to reduce the stress on the patient.

Priorities of respective multiple test items of an additional test order may be set based on the priorities of respective multiple possible lesions. The case where there are three possible lesions that are a possible lesion D1, a possible lesion D2 and a possible lesion D3 and a priority 2 is set in the possible lesion D1, a priority 1 is set in the possible lesion D2 and a priority 3 is set in the possible lesion D3 will be described as an example. In this case, test items are set for each of the possible lesions. In an example, the second outputting function 144 sets, as multiple test items of an additional test order, a test item D11, a test item D12 and a test item D13 for diagnosing the possible lesion D1; a test item D21, a test item D22 and a test item D23 for diagnosing the possible lesion D2; and a test item D31, a test item D32 and a test item D33 for diagnosing the possible lesion D3. Based on the priority 1 that is set in the possible lesion D2, priorities 1 to 3 are added respectively to the test item D21, the test item D22 and the test item D23. Based on the priority 2 that is set for the possible lesion D1, priorities 4 to 6 are added respectively to the test item D11, the test item D12 and the test item D13. Based on the priority 3 that is set in the possible lesion D3, priorities 7 to 9 are added respectively to the test item D31, the test item D32 and the test item D33. Redundant items among the test item D11, the test item D12, the test item D13, the test item D21, the test item D22, the test item D23, the test item D31, the test item D32 and the test item D33 may be omitted as appropriate.

The test practitioner carries out a test according to the additional test order and the second outputting function 144 acquires test results (step S111). The second outputting function 144 may register the acquired test results as previous data in the data storage apparatus 200.

The second outputting function 144 outputs the acquired test results (step S112). For example, the second outputting function 144 displays the acquired test results together with the test result that is processed at step S107 on the display 120. As for part of the test item, such as the test result of “1. history taking on history of exposure to asbestos”, the user is able to refer to the test result not via the second outputting function 144.

When the acquiring function 141 acquires multiple possible lesions, the first outputting function 143 may output a test result for each of the possible lesions at step S108. At step S112, the first outputting function 143 may further display a prevalence of each of the possible lesions. The prevalence may be general statistical information or may be a possibility that is calculated for each patient. For example, the first outputting function 143 calculates a prevalence for each patient and each lesion based on the test results that are acquired from the previous data and the test results that are acquired based on the additional test order and displays the prevalences on the display 120.

The user makes a diagnosis with reference to the test result resulting from the additional test order. For example, when the test result of “1. history taking on history of exposure to asbestos” clarifies that the patient had continued a work in which the patient is exposed to asbestos for many years, the user is able to diagnose “COPD due to exposure to asbestos for a long period”.

After outputting the test results at step S112, the second outputting function 144 moves to step S109 again and determines whether to create an additional test order. For example, when it is determined it is not possible to make a sufficient diagnosis based on the test results that are output at step S112, the user further inputs an additional test order. On the other hand, when the diagnosis completes, the second outputting function 144 determines that an additional test order is not necessary (NO at step S109) and ends the process. For example, when the user inputs “diagnosis determination”, the second outputting function 144 ends the process.

The flowchart illustrated in FIG. 2 is an example only and appropriate changes may be made. For example, step S102 may be omitted. In other words, the acquiring function 141 may omit searching a similar lesion. In this case, at step S103, the searching function 142 sets a test item based on the possible lesion that is acquired by the acquiring function 141 according to an input operation from the user.

At step S101, the acquiring function 141 may receive an input of symptoms of the patient instead of a possible lesion. In this case, at step S102, the acquiring function 141 searches for a possible lesion based on the input symptoms. The searching, for example, can be executed based on a table that determines a correspondence relationship between symptoms and lesions in advance. In an example, the acquiring function 141 receives an input of a symptom “couching” and performs searching and accordingly acquires possible lesions “asthma” and “COPD”.

As described above, according to the first embodiment, the acquiring function 141 acquires a possible lesion. Before executing a test according to a test order, the searching function 142 searches previous data for a relevant test item relevant to a test item of the test order. When the searching result contains a relevant test item, the first outputting function 143 acquires and outputs the test result that was collected in the past through the test of the relevant test item. When the searching result does not contain any relevant test item, the second outputting function 144 outputs an additional test order corresponding to the searching result. Accordingly, the ordering system 1 according to the first embodiment is able to efficiently utilize the previous test result. Accordingly, the ordering system is able to reduce the number of tests to be executed and thus reduce the stress on the patient and work of the user and at the same time shorten the workflow to diagnosis.

According to the first embodiment, when the searching result contains a relevant test item, the first outputting function 143 acquires a searching result that was collected in the past through the test of the relevant test item, processes the acquired test result into a form that matches the purpose of testing and outputs the processed test result. Thus, even when the previous test result has been stored in another diagnosis and treatment department or another hospital, the ordering system 1 according to the first embodiment is able to process the previous test result into a form that can be easily used for diagnosis and represent the processed test result.

According to the first embodiment, the first outputting function 143 extracts part of the test result that was collected in the past according to the user, processes the extracted test result into a form that matches the purpose of testing and outputs the processed test result. Thus, even when previous test result has been stored in another diagnosis and treatment department or another hospital, the ordering system 1 according to the first embodiment is able to omit unnecessary part of the previous test result and thus represent the reduced amount of test result. For example, when the previous test result has been stored in the department of cardiovascular internal medicine and the user is a doctor of the department of respiratory medicine, the ordering system 1 is able to represent the test result from which a part unnecessary to diagnose a respiratory organ has been omitted.

In the above-described first embodiment, the flowchart in the case where the possible lesions are “1. asthma”, “2. COPD” and “3. interstitial lung disease” is described as an example. On the other hand, in a second embodiment, a modification of the flowchart will be described, exemplifying the case where the possible lesions are “1. thoracic spine compression fracture” and “2. thoracic disc herniation”.

The ordering system 1 according to the second embodiment has the same configuration as that of the ordering system 1 illustrated in FIG. 1. As for the aspect that the ordering system 1 has the same configuration as that descried in the first embodiment, description thereof will be omitted by denoting the same configuration with the same reference numbers and letters as those in FIG. 1.

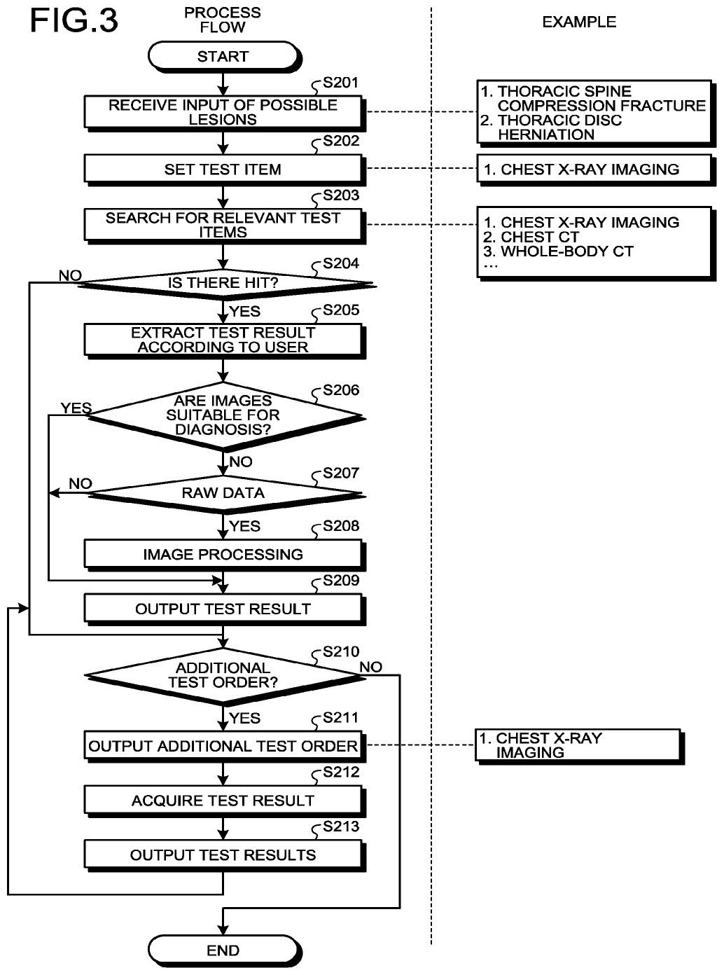

FIG. 3 is a flowchart for describing a process sequence of the ordering system 1 according to the second embodiment. First of all, the acquiring function 141 receives an input of possible lesions from the user (step S201). For example, as illustrated in FIG. 3, “1. thoracic spine compression fracture” and “2. thoracic disc herniation” are input as possible lesions. The acquiring function 141 may search for a similar lesion similar to the possible lesions that are input from the user. The acquiring function 141 may receive an input of symptoms of a patient instead of possible lesions. In this case, the acquiring function 141 searches for a possible lesion based on the input symptoms.

The searching function 142 then sets a test item based on the possible lesions that are acquired by the acquiring function 141 (step S202). For example, as illustrated in FIG. 3, the searching function 142 sets “1. chest X-ray imaging” as the test item. The searching function 142 then searches previous data for relevant test items relevant to the test item “1. chest X-ray imaging” (step S203). For example, as illustrated in FIG. 3, the searching function 142 searches the previous data for relevant test items, such as “1. chest X-ray imaging”, “2. chest CT” and “3. whole-body CT”.

The searching function 142 then determines whether there is a search hit (step S204). In other words, the searching function 142 determines whether the result of searching contains a relevant test item. When the result of searching by the searching function 142 contains a relevant test item (YES at step S204), the first outputting function 143 acquires a test result that was collected in the past in an test of the relevant test item and extracts part of the test result according to the user (step S205). The extraction process may be omitted as appropriate.

The first outputting function 143 processes the test result into a form that matches the purpose of testing and outputs the processed test result. Specifically, the first outputting function 143 processes the test result through the process of steps S206, S207 and S208 and outputs the processed test result at step S209.

Specifically, first of all, the first outputting function 143 determines whether the acquired test result is images that are suitable for diagnosis (step S206). For example, as illustrated in FIG. 3, in the case where the possible lesions are “1. thoracic spine compression fracture” and “2. thoracic disc herniation”, when the acquired test result is “X-ray image data that was collected in the past in the department of respiratory medicine”, the first outputting function 143 determines that the test result is not images that are not suitable for diagnosis. In other words, in general, image processing that is suitable for observation of respiratory organs is performed on “X-ray image data that was collected in the past in the department of respiratory medicine” and, because the X-ray image data tends to be unsuitable for observation of the thoracic spine, the first outputting function 143 determines that the test result is not images that are suitable for diagnosis.

When the acquired test result is not images that are unsuitable for diagnosis (NO at step S206), the first outputting function 143 determines whether there is raw data (step S207). In other words, the first outputting function 143 determines whether data that was collected by capturing images of the patient in the past and that is before the image generation processing is performed is left as previous data. When there is the raw data (YES at step S207), the first outputting function 143 acquires the raw data from the previous data and executes the image generation processing on the raw data, thereby generating X-ray image data (step S208). By performing image processing that is suitable for observation of the thoracic spine, the first outputting function 143 is able to generate X-ray image data suitable for diagnosis of “1. thoracic spine compression fracture” and “2. thoracic disc herniation”.

The first outputting function 143 outputs the test result (step S209). For example, when the test result that is acquired from the previous data is images suitable for diagnosis (YES at step S206), the first outputting function 143 displays the acquired X-ray image data on the display 120. When the test result that is acquired from the previous data is not images suitable for diagnosis but there is no raw data (NO at step S207), the first outputting function 143 displays the acquired X-ray image data on the display 120. For example, when there is raw data (YES at step S207), the first outputting function 143 displays X-ray image data that is generated based on the raw data that is acquired from the previous data on the display 120.

The second outputting function 144 then determines whether to create an additional test order (step S210). The second outputting function 144 may determine necessity of an additional test order automatically or according to an input operation from the user. For example, the user refers to the X-ray image data that is output at step S209 and checks whether there is a chest compression fracture. When no chest compression fracture is confirmed, the user makes an operational input indicating that an additional test order is necessary.

When it is determined that an additional test order is necessary (YES at step S210), the second outputting function 144 creates and outputs an additional test order (step S211). The test practitioner carries out tests according to the additional test order and the second outputting function 144 acquires a test result (step S212). The second outputting function 144 may register the acquired test result as previous data in the data storage apparatus 200.

The second outputting function 144 outputs the acquired test results (step S213). For example, the second outputting function 144 displays the acquired test result together with the test result that is acquired from the previous data (the test result that is output at step S209) on the display 120. The user makes a diagnosis while referring to the test result resulting from the additional test order.

For example, when the X-ray image data that is acquired from the previous data are images that were captured half a year ago and a chest compression fracture cannot be confirmed in the X-ray image data and a chest compression fracture can be confirmed in the X-ray image data that is acquired from the additional test order, the user is able to diagnose “a thoracic spine compression fracture having occurred in the last half a year”. In other words, the user is able to diagnose a thoracic spine compression fracture and estimate a rough time of incidence of the fracture.

After outputting the test result at step S213, the second outputting function 144 moves to step S210 again and determines whether to create an additional test order. In an example, when it is determined that a sufficient diagnosis cannot be made based on the test results that are output at step S213, the user further inputs an additional test order. In an example, the user inputs a test item “2. thoracic spine magnetic resonance imaging (MRI)” of an additional test order. On the other hand, when the diagnosis completes, the second outputting function 144 determines that no additional test order is necessary (NO at step S210) and ends the process. For example, when the user inputs “diagnosis determination”, the second outputting function 144 ends the process.

The setting function 145 may set a retention period for the raw data and the X-ray image data. In other words, the setting function 145 may set a retention period for the raw data that was collected in the past by capturing images of the patient with the X-ray diagnostic apparatus and the X-ray image data that is generated by executing the image generation processing on the raw data. The setting function 145 may uniformly set a retention period per facility, such as a hospital, may uniformly set a retention period per diagnosis and treatment department, or may set a retention period per patient. For example, the setting function 145 sets a retention period according to a lesion of a patient such that the retention period increases as the possibility of re-emergence of the lesion increases.

In a third embodiment, a modification of the flowchart will be described, exemplifying the case where the possible lesions are “1. hepatic cirrhosis” and “2. liver cancer”. The ordering system 1 according to the third embodiment has the same configuration as that of the ordering system 1 illustrated in FIG. 1. As for the aspect that the ordering system 1 has the same configuration as that descried in the first and second embodiments, description thereof will be omitted by denoting the same configuration with the same reference numbers and letters as those in FIG. 1.

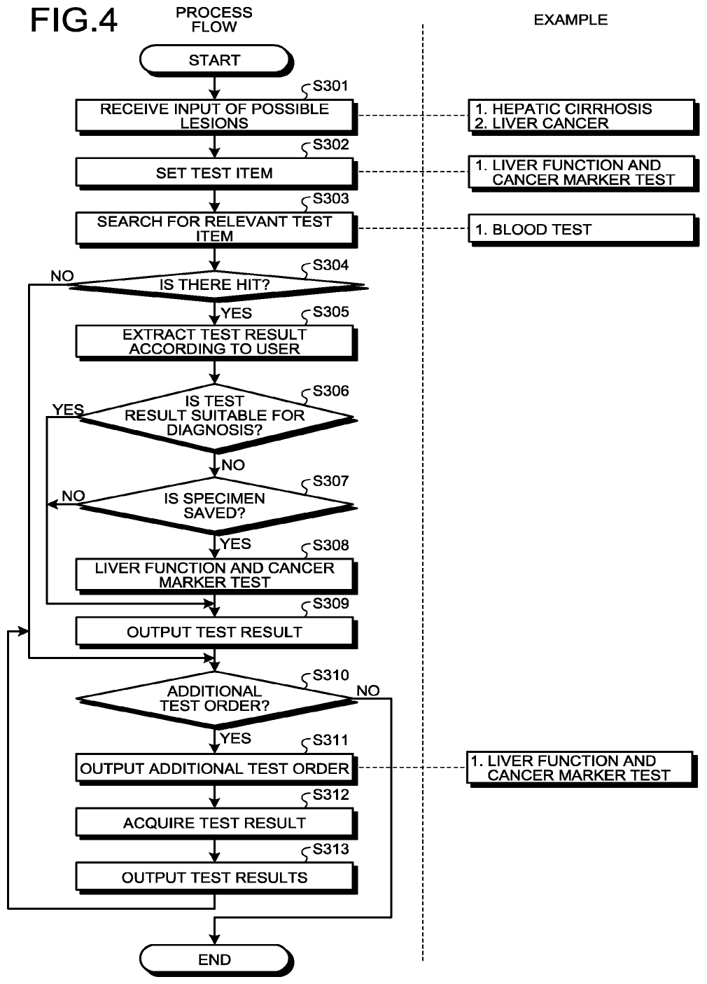

FIG. 4 is a flowchart for describing a process sequence of the ordering system 1 according to the third embodiment. First of all, the acquiring function 141 receives an input of possible lesions from the user (step S301). For example, as illustrated in FIG. 3, the user inputs “1. hepatic cirrhosis” and “2. liver cancer” as possible lesions. The acquiring function 141 may search for a similar lesion similar to the lesions that are input by the user. The acquiring function 141 may receive an input of symptoms of a patient instead of possible lesions. In this case, the acquiring function 141 searches for a possible lesion based on the input symptoms. The searching function 142 sets a test item based on the possible lesions that are acquired by the acquiring function 141 (step S302). For example, as illustrated in FIG. 4, the searching function 142 sets “1. liver function and cancer marker test” as a test item. The searching function 142 then searches previous data for a relevant test item relevant to the test item “1. liver function and cancer marker test” (step S303). For example, as illustrated in FIG. 4, the searching function 142 searches the previous data for a relevant test item “1. blood test”. In other words, the searching function 142 searches the previous data for “1. blood test” covering “1. liver function and cancer marker test” as a relevant test item relevant to “1. liver function and cancer marker test”.

The searching function 142 then determines whether there is a search hit (step S304). In other words, the searching function 142 determines whether the result of searching contains a relevant test item. When the result of searching by the searching function 142 contains a relevant test item (YES at step S304), the first outputting function 143 acquires a test result that was collected in the past by performing a test of the relevant test item and extracts part of the test result according to the user (step S305). The extraction processing may be omitted as appropriate.

The first outputting function 143 processes the test result into a form that matches the purpose of testing and outputs the processed test result. Specifically, the first outputting function 143 processes the test result through the processing of steps S306, S307 and S308 and outputs the processed test result at step S309.

Specifically, first of all, the first outputting function 143 determines whether the acquired test result is a test result that is suitable for diagnosis (step S306). For example, as illustrated in FIG. 4, when the possible lesions are “1. hepatic cirrhosis” and “2. liver cancer”, a test result of a liver function and cancer marker test among the blood test is suitable for diagnosis. When the acquired test result is a test result of a blood test different in type from the liver function and cancer marker test, the first outputting function 143 determines that it is not a test result suitable for diagnosis.

When the acquired test result is not a test result suitable for diagnosis (NO at step S306), the first outputting function 143 determines whether the specimen has been retained (step S307). For example, the first outputting function 143 determines whether blood that was drawn from the patient in a previous blood test has been retained. When the blood has been retained (YES at step S307), the first outputting function 143 executes the liver function and cancer marker test based on the retained blood and acquires a test result of the liver function and cancer marker test (step S308). In other words, the first outputting function 143 acquires processed data obtained by processing the specimen that was collected from the patient in the past according to the purpose of testing. The liver function and cancer marker test may be executed according to an instruction that is automatically issued by the first outputting function 143 to a specimen testing system or may be executed according to an instruction that is made by the user to the specimen testing system.

The first outputting function 143 outputs a test result (step S309). For example, when the test result that is acquired from the previous data is a test result that is suitable for diagnoses (YES at step S306), the first outputting function 143 displays the acquired test result on the display 120. In an example, when the test result that is acquired from the previous data is a test result of the liver function and cancer marker test, the first outputting function 143 displays the test result of the liver function and cancer marker test that is acquired from the previous data on the display 120. For example, when the test result acquired from the previous data is not a test result suitable for diagnosis and the specimen is not saved (NO at step S307), the first outputting function 143 displays the acquired test result on the display 120. When the specimen has been retained (YES at step S307), the first outputting function 143 displays the test result of the liver function and cancer marker test that is executed based on the retained blood on the display 120. In other words, the first outputting function 143 outputs the processed data obtained by processing the specimen that was collected from the patient in the past according to the purpose of testing.

The second outputting function 144 determines whether to create an additional test order (step S310). The second outputting function 144 may determine whether to create an additional test order automatically or according to an input operation from the user. For example, the user refers to the test result that is output at step S309 and checks whether there is an abnormality in the liver function. When no abnormality in the liver function is confirmed, the user makes an operational input indicating that an additional test order is necessary.

When it is determined that an additional test order is necessary (YES at step S310), the second outputting function 144 creates and outputs an additional test order (step S311). For example, as illustrated in FIG. 4, the second outputting function 144 creates and outputs an additional test order containing “1. liver function and cancer marker test”. The test practitioner carries out a test according to the additional test order and the second outputting function 144 acquires a test result (step S312). The second outputting function 144 may register the acquired test result and retention information in the data storage apparatus 200 as previous data. For example, the second outputting function 144 stores data indicating the result of the liver function and cancer marker test in the data storage apparatus 200. For example, the second outputting function 144 stores retention information on blood that is collected in the test carried out according to the additional test order in the data storage apparatus 200.

The second outputting function 144 outputs the acquired test result (step S313). For example, the second outputting function 144 displays the acquired test result on the display 120 together with the test result that is acquired from the previous data (the test result that is output at step S309). The user makes a diagnosis while referring to the test result resulting from the additional test order.

For example, when no abnormality in the liver function is confirmed in the test result of the liver function and cancer marker test that is acquired from the previous data and an increase of a liver cancer marker is confirmed in the test result of the liver function and cancer marker test that is acquired according to the additional test order, the user is able to diagnose “liver cancer”. Furthermore, the liver cancer had not occurred when the liver function and cancer marker test was executed in the past and thus the user is able to narrow down the degree of progress of liver cancer according to the time when the previous liver function and cancer marker test was executed and prepare a treatment plan.

After outputting the test results at step S313, the second outputting function 144 moves to step S310 again and determines whether to create an additional test order. For example, when it is determined that a sufficient diagnosis cannot be made based on the test results that are output at step S313, the user further inputs an additional test order. In an example, the user inputs a test item “2. abdominal ultrasonography” of the additional test order in order to perform detailed observation on the abdomen of the patient. On the other hand, when the diagnosis completes, the second outputting function 144 determines that no additional test order is necessary (NO at step S310) and ends the process. For example, when the user inputs “diagnosis determination”, the second outputting function 144 ends the process.

The setting function 145 may set a retention period for the specimen, such as blood, that is collected from the patient. The setting function 145 may uniformly set a retention period per facility, such as a hospital, may uniformly set a retention period per diagnosis and treatment department, or may set a retention period per patient. For example, the setting function 145 sets a retention period according to a lesion of a patient such that the retention period increases as the possibility of re-emergence of the lesion increases.

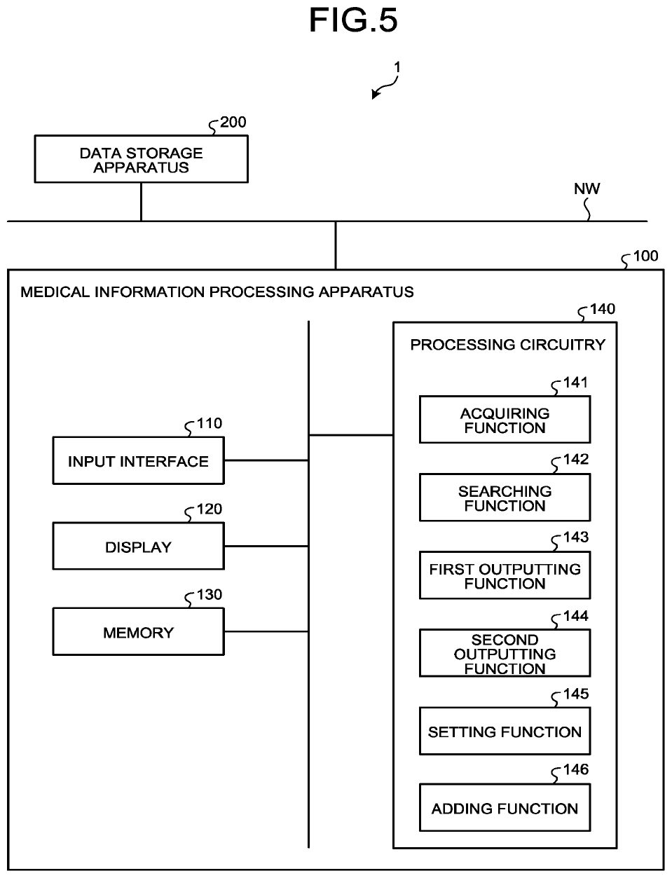

In a fourth embodiment, the case where previous data is searched based on anatomical features contained in image data will be described. The ordering system 1 according to the fourth embodiment is different in that, as illustrated in FIG. 5, the processing circuitry 140 further executes an adding function 146. FIG. 5 is a block diagram illustrating an exemplary configuration of the ordering system 1 according to the fourth embodiment. The adding function 146 is an exemplary adding unit. As for the aspect that the ordering system 1 has the same configuration as that descried in the first to third embodiments, description thereof will be omitted by denoting the same configuration with the same reference numbers and letters as those in FIG. 1.

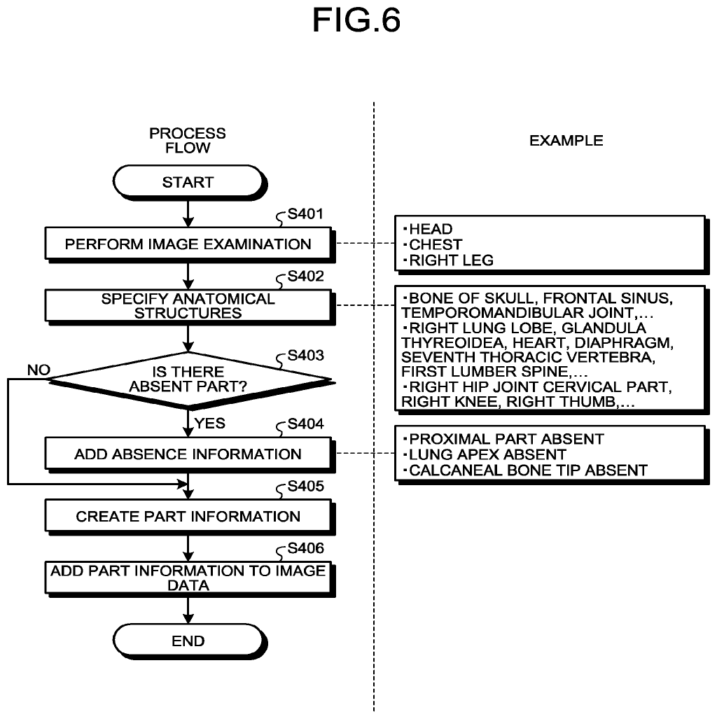

The adding function 146 adds part information representing an anatomical structure to image data that is saved in the data storage apparatus 200. A process of adding part information will be described using FIG. 6. FIG. 6 is a flowchart for describing a process sequence to add part information according to the fourth embodiment.

For example, as illustrated in FIG. 6, first of all, image examination on the patient is performed (step S401). Specifically, a modality collects raw data by capturing images of a part, such as the head, chest or the right leg, and executes image generation processing on the collected raw data, thereby generating image data. Examples of the modality include, for example, an X-ray diagnostic apparatus, an X-ray CT apparats, an MRI apparatus, an ultrasound diagnostic apparatus, a positron emission tomography (PET) apparatus, a single photon emission computed tomography (SPECT), or the like. The user then diagnoses the patient based on the collected image data.

The adding function 146 specifies an anatomical structure contained in the image data that is collected in the examination at step S401 (step S401). For example, when image data on the head of the patient is collected, the adding function 146 specifies anatomical structures, such as “the bone of the skull”, “the frontal sinus” and “the temporomandibular joint”. For example, when the image data on the chest of the patient is collected, the adding function 146 specifies anatomical structures, such as “the right lung lobe”, “the glandula thyreoidea”, “the heart”, “the diaphragm”, “the seventh thoracic vertebra”, and “the first lumbar spine”. For example, when image data on the right leg of the patient is collected, the adding function 146 specifies anatomical structures, such as “the right hip joint cervical part”, “the right knee” and “the right thumb”.

In an example, the adding function 146 aligns the collected image data and a human body model in which the positions of the anatomical structures are determined, thereby specifying anatomical structures contained in the image data. When the collected image data is three-dimensional data, the adding function 146 hierarchically specifies anatomical structures contained in the image data. In an example, when the collected image data is volume data that is collected by an X-ray CT apparatus, the adding function 146 aligns the volume data with a three-dimensional human body model and specifies anatomical structures contained in the image data per cross-sectional image (slice).

The adding function 146 determines whether there is an absent part (step S403). For example, the proximal end may be absent when image data on the head of the patient is collected, the apex of the lung may be absent when image data on the chest of the patient is collected, and the tip of the calcaneal bone may be absent when image data on the right leg of the patient is collected. For example, when image data on the chest of the patient is collected, the humeral center and distal parts may be absent while the humeral proximal part is contained in the image data.

When there are absent parts (YES at step S403), the adding function 146 adds absence information indicating the absent parts (step S404). For example, the adding function 146 adds absence information, such as “the proximal part absent”, “the lung apex absent”, and “the calcaneal bone tip absent”, to the anatomical structures that are specified at step S402. When there is not any absent part (NO at step S403), the processing at step S404 is omitted.

The adding function 146 then creates part information indicating the anatomical structures (step S405). For example, the part information is a list of the anatomical structures contained in the image data. When it is determined at step S43 that there is an absent part, the part information contains absent information. The adding function 146 adds the created part information to image data (step S406) and saves the image data to which the part information is added in the data storage apparatus 200 and then ends the process. When the image data is three-dimensional data, the adding function 146 hierarchically adds part information. In an example, when the collected image data is volume data that is collected by the X-ray CT apparatus, the adding function 146 adds part information to each slice.

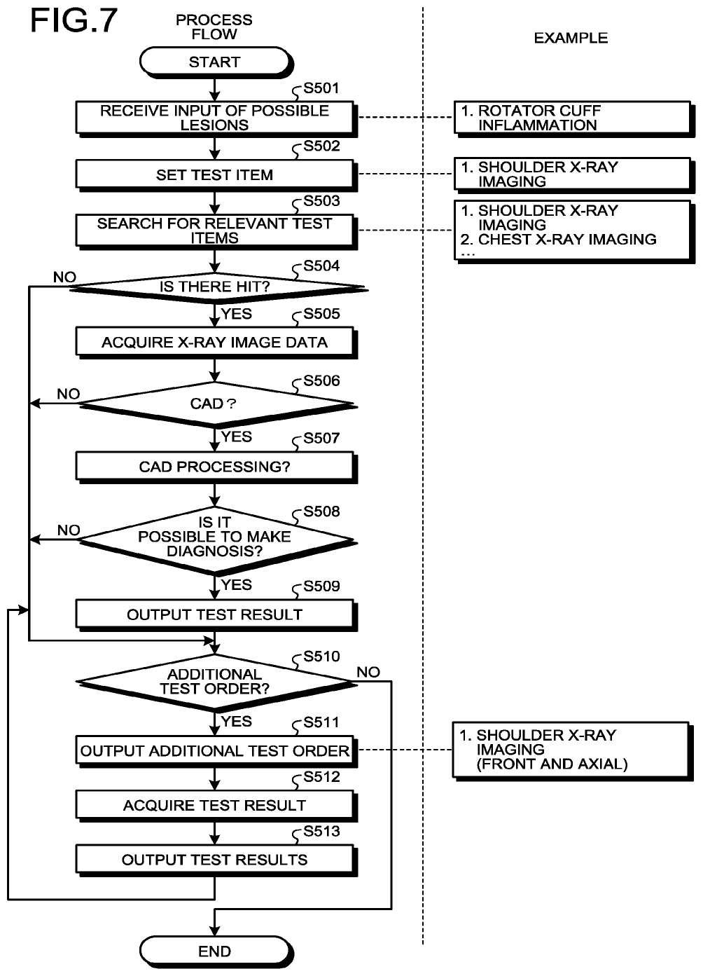

Using FIG. 7, a process sequence of the ordering system 1 will be described. FIG. 7 is a flowchart for describing the process sequence of the ordering system 1 according to the fourth embodiment.

First of all, the acquiring function 141 receives an input of a possible lesion from the user (step S501). For example, as illustrated in FIG. 7, the user inputs “1. rotator cuff inflammation” as a possible lesion. The acquiring function 141 may search for a similar lesion similar to the possible lesion that is input by the user. The acquiring function 141 may receive an input of symptoms of a patient instead of a possible lesion. In this case, the acquiring function 141 searches for a possible lesion based on the input symptoms.

The searching function 142 sets a test item based on the possible lesion that is acquired by the acquiring function 141 (step S502). For example, as illustrated in FIG. 7, the searching function 142 sets “1. shoulder X-ray imaging” as a test item. The searching function 142 searches previous data for a relevant test item relevant to the test item “1. shoulder X-ray imaging” (step S503). For example, as illustrated in FIG. 7, the searching function 142 searches the previous data for relevant test items, such as “1. shoulder X-ray imaging” and “2. chest X-ray imaging”.

The searching function 142 determines whether there is a search hit (step S504). In other words, the searching function 142 determines whether the result of searching contains a relevant test item. When the searching result contains relevant test items (YES at step S504), the first outputting function 143 acquires X-ray image data that was collected in the past through a test of the relevant test item (step S505). For example, the first outputting function 143 acquires chest X-ray image data that was collected in the past through the chest X-ray imaging. In an example, the first outputting function 143 acquires multiple sets of chest X-ray image data that were collected by performing chest simple imaging that was performed regularly in the past.

The first outputting function 143 processes the acquired chest X-ray image data into a form that matches the purpose of testing and outputs the processed X-ray image data. Specifically, the first outputting function 143 executes image processing corresponding to the purpose of testing on the chest X-ray image data through the process of the following steps S506, S507 and S508 and outputs the image data that is generated by performing the image processing at step S509.

For example, first of all, the first outputting function 143 determines whether CAD processing for detecting a disorder of the shoulders is executable on the acquired test result (step S506). In an example, when the acquired chest X-ray image data does not contain the shoulders, the first outputting function 143 determines that CAD processing for detecting a disorder of the shoulders is not executable. The first outputting function 143 is able to determine whether the chest X-ray image data contains the shoulders based on part information that is added to the chest X-ray image data. In another example, when it is not possible to use a CAD application, the first outputting function 143 determines that CAD processing is not executable.

When it is determined that CAD processing is executable, the first outputting function 143 executes CAD processing on the chest X-ray image data (step S507). For example, the first outputting function 143 executes image processing that is suitable for observation of bones and ligaments on the chest X-ray image data and executes CAD processing on the chest X-ray image data on which the image processing has been executed, thereby detecting a disorder of the shoulders.

The first outputting function 143 determines whether it is possible to make a diagnosis based on the result of CAD processing (step S508). In other words, depending on the imaging angle of the chest X-ray image data and image processing that was executed in the past on the chest X-ray image data, it may be not possible to detect a disorder of the shoulder appropriately by CAD processing. The first outputting function 143 determines whether it is possible to make a diagnosis based on the result of CAD processing and, when it is determined that it is possible to make a diagnosis, outputs a test result (step S509). For example, the first outputting function 143 displays, as the test result, image data in which the result of CAD processing is represented on the chest X-ray image data on the display 120. In other words, the first outputting function 143 outputs, as the test result, image data that is generated by executing image processing corresponding to the purpose of testing on the chest X-ray image data. The determination process of step S508 may be omitted as appropriate.

When the result of searching by the searching function 142 contains no relevant test item (NO at step S504), when CAD processing is not executable (NO at step S506), when it is not possible to make a diagnosis based on the result of CAD processing (NO at step S508), or after the test result is output at step S509, the second outputting function 144 determines whether to create an additional test order (step S510).

The second outputting function 144 may determine necessity of an additional test order automatically or according to an input operation from the user. For example, the user refers to the test result that is output at step S509 and checks whether there is an abnormality in the shoulder ligaments. When no abnormality in the shoulder ligaments is confirmed, the user makes an operational input indicating that an additional test order is necessary.

When it is determined that an additional test order is necessary (YES at step S510), the second outputting function 144 creates and outputs an additional test order (step S511). For example, as illustrated in FIG. 7, the second outputting function 144 creates and outputs an additional test order containing “1. shoulder X-ray imaging (front and axial)”. The test practitioner carries out a test according to the additional test order and the second outputting function 144 acquires a test result (step S512). In an example, the second outputting function 144 acquires shoulder X-ray image data that is collected according to the additional test order and executes CAD processing on the acquired shoulder X-ray image data, thereby acquiring image data representing the result of CAD processing. The second outputting function 144 may register the acquired test result as previous data in the data storage apparatus 200. For example, the second outputting function 144 stores image data representing the result of CAD processing in the data storage apparatus 200.

The second outputting function 144 outputs the acquired test result (step S513). For example, the second outputting function 144 displays the acquired test result on the display 120 together with the test result that is acquired from the previous data (the test result that is output at step S509). The user makes a diagnosis while referring to the test result resulting from the additional test order. When the diagnosis completes, the second outputting function 144 determines that an additional test order is unnecessary (NO at step S510) and ends the process. For example, when the user inputs “diagnosis determination”, the second outputting function 144 ends the process.

In an example, at step S509, the user refers to the results of CAD processing based on the multiple sets of chest X-ray image data that were regularly collected in the past and confirms that ligament calcification has gradually got greater since a year ago. In this case, the user provides appropriate treatments according to the result of checking and make a diagnosis indicating that shoulder X-ray imaging will be performed regularly. When chest X-ray imaging will be performed regularly, part of shoulder X-ray imaging that will be performed may be replaced with chest X-ray imaging. The second outputting function 144 will regularly perform shoulder X-ray imaging and, when the times of imaging overlap, create a test order indicating that the shoulder X-ray imaging is replaced with chest X-ray imaging such that a test reservation will reflect the test order.

As for FIG. 6 and FIG. 7, it is described that part information is added to image data and the first outputting function 143 processes the image data that is acquired as the test result based on the part information and outputs the processed image data. Embodiments are however not limited thereto, and the adding function 146 may add part information to data that was collected in the past by capturing images of the patient. In other words, the adding function 146 may add part information to raw that is before the image generation processing is performed. In this case, the first outputting function 143 may acquire raw data or image data as the test result and process the raw data or image data based on the part information and output the processed data.

As described above, according to the fourth embodiment, the adding function 146 adds the part information to the raw data that was collected in the past by capturing images of the patient or to the image data that is generated by executing the image generation processing on the raw data. The first outputting function 143 processes the raw data or the image data that is acquired as the test result based on the part information and outputs the processed data. Accordingly, the ordering system 1 according to the first embodiment is capable of easily determining whether the raw data that is acquired from the previous data or the image data contains a part necessary for the purpose of testing easily based on the part information, thereby shortening the processing time.

The first to fourth embodiments have been described and various different modes may be carried out in addition to the above-described embodiments.

The above-described embodiments have been given as ones in which the test result that was collected in the past is processed and output by the first outputting function 143. Embodiments however are not limited thereto. For example, processing on the test result that was collected in the past may be performed outside the facility and the first outputting function 143 may acquire and output the processed test result.

When a specimen, such as blood, that was collected from the patient in the past is retained in an external facility, it takes time and costs to transfer the specimen to a facility in which the medical information processing apparatus 100 and a user are situated. Thus, the external facility in which the specimen is retained executes processing on the specimen and the first outputting function 143 acquires processed data obtained by processing the specimen in the external facility, which enables efficient processing on the specimen.

In an example, first of all, the searching function 142 searches previous data for a relevant test item relevant to a test item of the current test order. The first outputting function 143 then acquires retention information on the specimen that was collected in the past through a test of the relevant test item that is searched for by the first outputting function 143. When the specimen is retained in the external facility, the first outputting function 143 issues an instruction for a test on the specimen according to the current purpose of testing to the external facility retaining the specimen and acquires processed data. For example, the first outputting function 143 issues an instruction for executing a liver function and cancer marker test based on the retained blood to the external facility retaining the specimen and acquires data of the test result of the liver function and cancer marker test via the network NW.

In general, compared to image data, raw data that is before the image generation processing is performed has a large data size. For this reason, when raw data that was collected from a patient in the past is retained in an external facility, it takes time and costs to transmit the raw data to a facility in which the medical information processing apparatus 100 and a user are situated. Thus, the external facility retaining the raw data executes the image generation processing on the raw data and the first outputting function 143 acquires the image data that is generated in the external facility, which enables efficient processing on the raw data.

In an example, first of all, the searching function 142 searches previous data for a relevant test item relevant to a test item of the current test order. The first outputting function 143 then acquires information on retention of raw data that was collected in the past through a test of the relevant test item that is searched for by the searching function 142. For example, the first outputting function 143 acquires retention information, such as a place where the raw data that was collected in the past is retained and a period during which the raw data is retained. When raw data is retained in an external facility, the first outputting function 143 issues an instruction for image processing according to the current purpose of testing to the external facility retaining the raw data and acquires image data that is processed into a form that matches the current purpose of testing. For example, when the current purpose of testing is diagnosing the lungs, the first outputting function 143 issues an instruction for executing the image generation processing and various types of image processing for generating image data suitable for observing the lungs to the external facility retaining the raw data and acquires image data via the network NW.

In the above-described embodiments, the user, such as a doctor, inputs a possible lesion or symptoms. Embodiments however are not limited thereto. For example, a user may input a possible lesion or a symptom. In this case, the user is able to predict a disorder, searches for a hospital capable of carrying out necessary tests, and select a hospital or a doctor based on test results that were collected with respect to the user in the past.

In the above-described embodiment, the medical information processing apparatus 100 is described as one that issues a test order. In other words, the medical information processing apparatus 100 is described as an ordering apparatus. Embodiments however are not limited thereto. An ordering apparatus may be arranged independently of the medical information processing apparatus 100. In that case, the ordering apparatus executes a function corresponding to the second outputting function 144 of The medical information processing apparatus 100.

For example, the searching function 142 acquires a test order from the ordering apparatus and searches previous data for a relevant test item relevant to a test item of the acquired test order. The first outputting function 143 acquires a test result that was collected in the past through a test of a relevant test item contained in the result of searching, processes the test result into a form matching the current purpose of testing of the test order, and outputs the processed test result. The ordering apparatus outputs an additional test order according to the result of searching by the searching function 142.

The word “processor” used in the descriptions given above refers to, for example, a central processing unit (CPU), a graphics processing unit (GPU), or a circuit, such as an application specific integrated circuit (ASIC), a programmable logic device (for example, a simple programmable logic device (SPLD), a complex programmable logic device (CPLD) or a field programmable gate array (FPGA)). The processor reads programs that are saved in the memory 130 and executes the programs, thereby implementing the functions.

FIG. 1 illustrates that the single memory 130 stores the programs corresponding to the respective processing functions. Embodiments are however not limited thereto. For example, a plurality of memories 130 may be arranged in a distributed manner and the processing circuitry 140 may be configured to read the corresponding program from the individual memory 130. Instead of saving the programs in the memory 130, the programs may be directly incorporated in the circuit of the processor. In this case, the processor reads the programs that are incorporated in the circuit and executes the programs, thereby implementing the functions.

The processing circuitry 140 may implement the functions using a processor of an external apparatus that is connected via a network. For example, the processing circuitry 140 reads a program corresponding to each function from the memory 130 and executes the program and uses a group of servers that is connected with the medical information processing apparatus 100 via a network (clouds) as computation resources, thereby implementing each function illustrated in FIG. 1.

Each of the components of each of the devices according to the above-described embodiments is a functional idea and thus need not necessarily be configured physically as unillustrated in the drawings. In other words, specific modes of distribution and integration of the devices are not limited to those illustrated in the drawings, and all or part of the devices may be configured in a distributed or integrated manner functionally or physically in any unit according to various types of loads and the situation in which the devices are used. Furthermore, all or any part of the processing functions implemented by the devices may be implemented by a CPU and programs that are analyzed and executed by the CPU or may be implemented as wired logic hardware.

The processing method described in the above-described embodiments can be implemented by executing a program prepared in advance with a computer, such as a personal computer or a work station. The program is distributable via a network, such as the Internet. The program may be recorded in a computer-readable recording medium, such as a hard disk, a flexible disk (FD), a CD-ROM, a MO or a DVD) and may be read from the recording medium by the computer and thus executed.

According to at least one of the above-described embodiments, it is possible to efficiently utilize previous test results.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to,
acquire a possible lesion;
before executing a test according to a test order corresponding to the possible lesion, searches previous data for a relevant test item relevant to a test item of the test order;
when a result of searching contains the relevant test item, acquire a test result that is collected through a test of the relevant test item, process the acquired test result into a form that matches a purpose of testing of the test order, and output the processed test result; and
when the result of searching does not contain the relevant test item, output an additional test order corresponding to the result of searching.

2. The medical information processing apparatus according to claim 1, wherein the processing circuitry is configured to, when the result of searching contains the relevant test item relevant to part of the test item,
process the test result that was collected in the past through the test of the relevant test item into a form that matches the purpose of testing and output the processed test result, and
output the additional test order for executing the test of the test item whose corresponding relevant test item relevant to the test item is not contained in the result of searching.

3. The medical information processing apparatus according to claim 1, wherein the processing circuitry is configured to acquire multiple test results that were collected in the past and add priorities to the acquired multiple test results, respectively, process the test results into a form that matches the purpose of testing and outputs the processed test results.

4. The medical information processing apparatus according to claim 1, wherein the processing circuitry is configured to add priorities to multiple test items of the additional test order and output the examination items respectively with the priorities.

5. The medical information processing apparatus according to claim 1, wherein the processing circuitry is configured to acquire the test result that was collected in the past through the test of the relevant test item, extract part of the acquired test result according to a user, process the extracted test result into a form that matches the purpose of testing, and output the processed test result.

6. The medical information processing apparatus according to claim 1, wherein the processing circuitry is configured to output, as the test result, processed data obtained by processing a specimen that was collected from a patient in the past according to the purpose of testing.

7. The medical information processing apparatus according to claim 6, wherein the processing circuitry is configured to further set a period during which the specimen is retained.

8. The medical information processing apparatus according to claim 1, wherein the processing circuitry is configured to acquire data that was collected by capturing an image of a patient in the past or image data that is generated by executing image generation processing on the data and output image data that is generated by executing image processing corresponding to the purpose of examination on the data or the image data.

9. The medical information processing apparatus according to claim 8, wherein the processing circuitry is configured to further set a period during which data that was collected by capturing an image of a patient in the past or image data that is generated by executing image generation processing on the data is retained.

10. The medical information processing apparatus according to claim 8, wherein the processing circuitry is configured to
add, to the data that was collected by capturing an image of a patient in the past or the image data that is generated by executing the image generation processing on the data, part information representing an anatomical structure contained in the data or the image data, and
process the data or the image data that is acquired as the test result based on the part information and output the processed data or image data.

11. The medical information processing apparatus according to claim 10, wherein the processing circuitry is configured to, when the data or the image data is three-dimensional data, hierarchically add the part information.

12. The medical information processing apparatus according to claim 1, wherein the processing circuitry is configured to search the previous data for the relevant test item that is stored in each of multiple diagnosis and treatment departments.

13. The medical information processing apparatus according to claim 1, wherein the processing circuitry is configured to search the previous data that is stored in each of multiple facilities for the relevant test item.

14. The medical information processing apparatus according to claim 1, wherein the processing circuitry is configured to, when multiple possible lesions are acquired, add priorities respectively to multiple test items of multiple additional test orders corresponding respectively to the multiple possible lesions and output the test items respectively with the priorities.

15. The medical information processing apparatus according to claim 1, wherein the processing circuitry is configured to display a prevalence of the possible lesion.

16. An ordering system comprising a processing circuitry configured to,
acquire a possible lesion;
before executing a test according to a test order corresponding to the possible lesion, searches previous data for a relevant test item relevant to a test item of the test order;
when a result of searching contains the relevant test item, acquire a test result that is collected through a test of the relevant test item, process the acquired test result into a form that matches a purpose of testing of the test order, and output the processed test result; and
when the result of searching does not contain the relevant test item, output an additional test order corresponding to the result of searching.

17. A method comprising:
acquiring a possible lesion;
before executing a test according to a test order corresponding to the possible lesion, searching previous data for a relevant test item relevant to a test item of the test order;
when a result of searching contains the relevant test item, acquiring a test result that is collected through a test of the relevant test item, processing the acquired test result into a form that matches a purpose of testing of the test order, and outputting the processed test result; and
when the result of searching does not contain the relevant test item, outputting an additional test order corresponding to the result of searching.
